(19) United States
(12) Patent Application Publication
(10) Pub. No.: US 2007/0055115 A1
(43) Pub. Date: Mar. 8, 2007

(54) Characterization of sleep disorders using composite patient data

(76) Inventors: Jonathan Kwok, Shoreview, MN (US); Kent Lee, Shoreview, MN (US)

Correspondence Address: HOLLINGSWORTH & FUNK, LLC, Suite 125, 8009 34th Avenue South, Minneapolis, MN, 55425, US

(21) Appl. No.: 11/222,384

(22) Filed: Sep. 8, 2005

(51) Int. Cl. A61B 5/00; A61B 5/04; A61B 5/08

(52) U.S. Cl. 600/300; 600/509; 600/529; 128/920

(57) ABSTRACT

Systems and methods provide for evaluating sleep disorders, and involve implantably detecting one or more conditions associated with a sleep disorder, and receiving manually-reported patient data having relevance to the sleep disorder or patient condition. Using the detected conditions and the patient data, a quantitative diagnostic value for the sleep disorder is produced. The diagnostic value may be indicative of presence or non-presence of the sleep disorder or indicative of a level of severity of the sleep disorder. The manually-reported patient data may be acquired by use of a questionnaire or other patient question/answer facility.

### FIELD OF THE INVENTION

The present invention relates generally to detecting the presence of sleep disorders.

### BACKGROUND OF THE INVENTION

Sleep is generally beneficial and restorative to a patient, exerting great influence on the quality of life. The human sleep/wake cycle generally conforms to a circadian rhythm that is regulated by a biological clock. Regular periods of sleep enable the body and mind to rejuvenate and rebuild. The body may perform various tasks during sleep, such as organizing long term memory, integrating new information, and renewing tissue and other body structures.

Lack of sleep and/or decreased sleep quality may have a number of causal factors including, e.g., respiratory disturbances, nerve or muscle disorders, and emotional conditions, such as depression and anxiety. Chronic, long-term sleep-related disorders e.g., chronic insomnia, sleep-disordered breathing, and sleep movement disorders, including restless leg syndrome (RLS), periodic limb movement disorder (PLMD) and bruxism, may significantly affect a patient's sleep quality and quality of life.

Sleep apnea, for example, is a fairly common breathing disorder characterized by periods of interrupted breathing experienced during sleep. Sleep apnea is typically classified based on its etiology. One type of sleep apnea, denoted obstructive sleep apnea, occurs when the patient's airway is obstructed by the collapse of soft tissue in the rear of the throat. Central sleep apnea is caused by a derangement of the central nervous system control of respiration. The patient ceases to breathe when control signals from the brain to the respiratory muscles are absent or interrupted. Mixed apnea is a combination of the central and obstructive apnea types. Regardless of the type of apnea, people experiencing an apnea event stop breathing for a period of time. The cessation of breathing may occur repeatedly during sleep, sometimes hundreds of times a night and occasionally for a minute or longer.

In addition to apnea, other types of disordered respiration have been identified, including, for example, hypopnea (shallow breathing), dyspnea (labored breathing), hyperpnea (deep breathing), and tachypnea (rapid breathing). Combinations of the disordered respiratory events described above have also been observed. For example, Cheyne-Stokes respiration (CSR) is associated with rhythmic increases and decreases in tidal volume caused by alternating periods of hyperpnea followed by apnea and/or hypopnea. The breathing interruptions of CSR may be associated with central apnea, or may be obstructive in nature. CSR is frequently observed in patients with congestive heart failure (CHF) and is associated with an increased risk of accelerated CHF progression.

Movement disorders such as restless leg syndrome (RLS), and a related condition, denoted periodic limb movement disorder (PLMD), are emerging as one of the more common sleep disorders, especially among older patients. Restless leg syndrome is a disorder causing unpleasant crawling, prickling, or tingling sensations in the legs and feet and an urge to move them for relief. RLS leads to constant leg movement during the day and insomnia or fragmented sleep at night. Severe RLS is most common in elderly people, although symptoms may develop at any age. In some cases, it may be linked to other conditions such as anemia, pregnancy, or diabetes.

Many RLS patients also have periodic limb movement disorder (PLMD), a disorder that causes repetitive jerking movements of the limbs, especially the legs. These movements occur approximately every 20 to 40 seconds and cause repeated arousals and severely fragmented sleep.

An adequate duration and quality of sleep is required to maintain physiological homeostasis. Untreated, sleep disorders may have a number of adverse health and quality of life consequences ranging from high blood pressure and other cardiovascular disorders to cognitive impairment, headaches, degradation of social and work-related activities, and increased risk of automobile and other accidents.

### SUMMARY OF THE INVENTION

The present invention is directed to systems and methods for evaluating sleep disorders and, more particularly, to diagnosing sleep disorders. Embodiments of the invention are directed to implantably detecting one or more conditions associated with a sleep disorder, and receiving manually-reported patient data having relevance to the sleep disorder or patient condition. Using the detected conditions and the patient data, a quantitative diagnostic value for the sleep disorder is produced.

Other embodiments are directed to implantably sensing one or more conditions associated with a sleep disorder, and computing a detection value based on the sensed one or more conditions. Manually-reported patient data having relevance to the sleep disorder or patient condition is received, and a patient data score is computed using the received patient data. A diagnostic value for the sleep disorder is computed using the detection value and patient data score.

In various embodiments, the diagnostic value is indicative of presence or non-presence of the sleep disorder. For example, the diagnostic value may be a Boolean value, and producing the diagnostic value may involve logically combining the detection value and the patient data score to produce the diagnostic value. In other embodiments, the diagnostic value is indicative of a level of severity of the sleep disorder. For example, the diagnostic value may be a numerical value, and producing the diagnostic value involves mathematically combining the detection value and the patient data score to produce the diagnostic value.

Receiving the patient data may involve receiving the patient data in the form of a questionnaire. The questionnaire may, for example, include a number of questions each of which is assigned a value or other importance indicator. The patient data score may be computed by operating on the values, such as by summing the values (e.g., integers) associated with questions.

Computing the detection value may involve summing a number of the one or more conditions associated with the sleep disorder sensed over a predefined duration of time. Sensing the conditions may also involve detecting apnea or hypopnea events over a predefined duration of time, and computing the detection values may involve computing an apnea/hypopnea index (AHI) based on the number of apnea or hypopnea events detected over the predefined duration of time.

According to various embodiments, one or more patient condition indicators may be received, and one or more patient condition values corresponding to the one or more patient condition indicators may be produced. The diagnostic value may be produced using the detection value, patient data score, and the one or more patient condition values.

In other embodiments, a first threshold associated with detection of the one or more conditions associated with the sleep disorder is provided, and a second threshold associated with the patient data is provided. The detection value is computed using the sensed one or more conditions that exceed the first threshold, and the patient data score is computed using the received patient data that exceeds the second threshold.

Further aspects involve displaying one or more of the diagnostic value, patient data score, and detection data. Other aspects involve producing trend data using a plurality of the detection values computed over time, and producing alert information when the trend data exceeds one or more thresholds indicative of presence of the sleep disorder.

The diagnostic value may be produced by use of a networked system, a programmer, or other patient-external system. The patient data may be received via a programmer or a patient management system interface.

Further embodiments of the present invention are directed to an apparatus including a body implantable sensing device configured to sense one or more conditions associated with a sleep disorder. A user interface device is configured to receive manually-reported patient data having relevance to the sleep disorder or patient condition. A processing system is configured to compute a detection value based on the sensed one or more conditions, compute a patient data score using the received patient data, and produce a diagnostic value for the sleep disorder using the detection value and patient data score.

The body implantable sensing device may include an implantable cardiac monitoring device or an implantable cardiac energy delivery device. The body implantable sensing device may include a sleep disordered breathing sensor, and the detection value computed by the processing system may be an apnea/hypopnea index. The user interface device may include a programmer configured to communicatively couple to the body implantable sensing device. The user interface device may also include a network interface configured to communicatively couple to a patent management network system.

### BRIEF DESCRIPTION OF THE DRAWINGS

FIGS. 1A-1C illustrate flowcharts of methods for diagnosing sleep disorders in accordance with embodiments of the present invention;

FIG. 2 illustrates a flowchart of methods for diagnosing sleep disorders in accordance with embodiments of the present invention;

FIG. 3 illustrates a flowchart of a method for combining multiple diagnostics to yield a single diagnostic output indicative of presence, absence, or severity of a sleep disorder in accordance with embodiments of the present invention;

FIG. 4 is an illustration of a respiratory waveform representative of one of several types of physiologic signals that may be used to sense or detect a sleep disorder in accordance with embodiments of the present invention;

FIG. 5 illustrates apnea/hypopnea index trend data computed by use of an implantable medical device that may be useful in tracking progression of a sleep disorder over time in accordance with embodiments of the present invention;

FIG. 6 is an illustrative example of a questionnaire by which patient condition information may be manually reported for entry as data into a sleep disorder diagnosis system in accordance with embodiments of the present invention;

FIG. 7 is a block diagram of a diagnostic system configured to sense one or more physiologic parameters useful in detecting the presence and/or severity of sleep disorders;

FIG. 8 is an illustration of a cardiac rhythm management system that implements sleep disorder diagnostics in accordance with embodiments of the present invention; and

FIGS. 9-12 are respiratory waveforms that may be developed by a medical device implementing sleep disorder detection methodologies of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which are shown by way of illustration, various embodiments by which the invention may be practiced. It is to be understood that other embodiments may be utilized, and structural and functional changes may be made without departing from the scope of the present invention.

An adequate quality and quantity of sleep is required to maintain physiological homeostasis. Prolonged sleep deprivation or periods of highly fragmented sleep ultimately will have serious health consequences. Chronic fragmented sleep may be associated with various cardiac or respiratory disorders affecting a patient's health and quality of life.

By way of example, a significant percentage of patients between 30 and 60 years experience some symptoms of disordered breathing, primarily during periods of sleep. Sleep disordered breathing is associated with excessive daytime sleepiness, systemic hypertension, increased risk of stroke, angina and myocardial infarction. Disturbed respiration can be particularly serious for patients concurrently suffering from cardiovascular deficiencies. Disordered breathing is particularly prevalent among congestive heart failure patients, and may contribute to the progression of heart failure.

Assessment of sleep is traditionally performed in a polysomnographic sleep study at a dedicated sleep facility. Polysomnographic studies involve acquiring sleep-related data, including the patient's typical sleep patterns and the physiological, environmental, contextual, emotional, and other conditions affecting the patient during sleep. However, such studies are costly, inconvenient to the patient, and may not accurately represent the patient's typical sleep behavior.

Sleep assessment in a laboratory setting presents a number of obstacles in acquiring an accurate picture of a patient's typical sleep patterns including arousals and sleep disorders. For example, spending a night in a sleep laboratory typically causes a patient to experience a condition known as “first night syndrome,” involving disrupted sleep during the first few nights in an unfamiliar location. In addition, sleeping while instrumented and observed may not result in a realistic perspective of the patient's normal sleep patterns.

The present invention is directed to methods and systems for detecting the presence of sleep disorders and to producing diagnostic information concerning sleep disorders. Embodiments of the present invention employ an implantable or partially implantable device or sensor that is implemented to sense one or more conditions associated with a sleep disorder. In addition to the device-acquired information, patient-related data having relevance to the sleep disorder is acquired. The patient-related data is typically developed from patient or clinician commentary concerning patient condition, well-being, and/or patient history.

The patient-related data is combined with the device-acquired information to provide an enhanced evaluation of sleep disorders that may be adversely impacting the patient. The combined information may, for example, provide a physician with a binary diagnosis as to whether a particular sleep disorder is present or absent. The combined information may also provide a physician with an indication as to the severity of a detected sleep disorder. Use of device-acquired information in combination with manually-reported patent data has been found to improve detection and/or diagnosis of sleep disorders.

Patient medical systems in accordance with the present invention may be implemented to identify sleep disorders to a high degree of sensitivity and specificity. Sensitivity of a medical system enables the detection of a sleep disorder, and specificity of a medical system enables accurate identification of a sleep disorder so that benign conditions are not treated as sleep disorders. Patient medical systems implemented in accordance with the present invention provide for both increased sensitivity and specificity in identifying sleep disorders by combining manually-reported input patient data with implantably sensed sleep disorder diagnostic data. This combination has been found to yield a better sensitivity and specificity than sensed sleep disorder data or manually input data alone.

The following discussion is generally directed to embodiments of the invention that provide for enhanced detection and/or diagnosis of breathing disorders and, more particularly, to enhanced detection and/or diagnosis of apnea and hypopnea respiratory disorders. It is understood that the principles of the present invention may be implemented in methods and systems that provide for enhanced detection and/or diagnosis of other forms of sleep disorders, such as the various respiratory disorders discussed previously, and movement disorders, such as restless leg syndrome and periodic limb movement disorder, for example. Accordingly, the embodiments described below are provided for illustrative purposes, and are not to be regarded as limiting the scope of the present invention.

According to embodiments of the present invention, a sleep disorder diagnostic may be implemented in an implantable medical device. The device acquires information concerning one or more conditions related to one or more sleep disorders that may be afflicting the patient. The device-acquired data is transferred to a patient-external system, such as a programmer or user interface to a patient management system, for example. A detection value, such as an index value, is computed based on the device-acquired data.

At an appropriate time, such as during follow-up with the patient or clinician, a questionnaire or other form or type of question/answer facility is used to acquire patient data reported by the patient or clinician (often referred to herein as manually-reported patient data). The question/answer facility may be implemented in the programmer or patient management interface as an interactive questionnaire presented on a display, for example. The question/answer facility may also be implemented in a portable or home-based system or device.

Patients may, for example, input answers to questions presented in the questionnaire interface by themselves or via clinician assistance. The patient may provide such answers at their convenience or at a predetermined time or frequency (e.g., once every day). Patient answers may also be acquired at varying times of the day or at the same time each day the questionnaire facility is invoked. The questionnaire may also be invoked by a physician or clinician's request, either remotely by an appropriately configured patient management device or in person by use of a programmer. The questionnaire may use a standardized listing of questions (e.g., Epworth Sleepiness Scale Questionnaire, as described in Johns M W, Daytime Sleepiness, Snoring, and Obstructive Sleep Apnea. The Epworth Sleepiness Scale, Chest. 1993; 103:30-36), other questions concerning patient condition or well-being, or a proprietary listing of questions, for example.

After inputting patient answers into the question/answer facility, a questionnaire score (often referred to herein as a patient data score) is computed based on the patient answers input into the question/answer facility. The detection value developed using the device-acquired data and the questionnaire score developed using the question/answer facility are used to produce a quantitative output, such as a numerical or Boolean value, concerning the sleep disorder. The quantitative output value may indicate the presence/absence and/or severity of a sleep disorder.

In various embodiments, clinically significant decision thresholds may be used for the sleep disorder diagnosis. For example, all values greater than the threshold are indicative of a positive diagnosis for the disorder. Such thresholds and value/threshold comparisons can be made for both the device-acquired data and the questionnaire data.

The result of combining the device-acquired data and the questionnaire data that exceed their respective thresholds may be presented to the physician or patient as a binary diagnosis (presence or absence of the disorder), for example. One illustrative method of combining the data involves obtaining the binary diagnosis result for the device-acquired data and the questionnaire data, respectively, and passing the binary diagnosis results through a logical operator, such as a logical AND or a logical OR, for example.

The output from the logical operator represents a binary outcome for indicating the patient's sleep disorder severity. Alternatively, the device-acquired data and the questionnaire data may be manipulated by decision trees, neural networks, or other formulaic methods to generate a binary diagnosis or a scale of severity of the sleep disorder.

It may be desirable to include other patient condition information in the sleep disorder analysis. Such patient condition may include, for example, risk factor indicators, such as weight, neck size, hypertension, daily habits and activities, oximetry, and other questions a physician may ask of their patient in the diagnosis. Adding more sources of information to the analysis typically leads to a more accurate diagnosis. The various sources of information are typically analyzed separately (e.g., at different times and/or locations) and then the results of the various analyses are combined to form the diagnosis.

Various forms of output may be displayed to the physician and/or patient, including questionnaire score, average/trended device-acquired data values, a composite score based on the questionnaire score and the detection value developed from the device-acquired data, and/or diagnosis decision (e.g., presence or absence of a sleep disorder), among other information.

Detection methods and systems may be used for diagnostic purposes and/or to alert a patient or a clinician that a sleep disorder is present or likely to occur. Alternatively or additionally, the detection methods and systems may be used to form sleep disorder therapy decisions, such as by allowing clinicians to modify or initiate sleep disorder treatment in order to mitigate detected sleep disorders. Further, the detection of sleep disorders may also be used to automatically initiate disordered breathing therapy to prevent or mitigate a sleep disorder.

In an embodiment of the invention, detected and/or analyzed sleep disorder diagnostic information may be downloaded to an advanced patient management (APM) system from an implantable cardiac rhythm management (CRM) system, and an interactive questionnaire may be displayed at the APM system interface for a clinician to administer to a patient. The questionnaire results are analyzed after the patient's answers are entered, and the questionnaire score, average/trended sleep disorder value, and a composite score or decision may be displayed at the APM interface. This functionality may similarly be implemented using a programmer or other patient-external system. The combination of the analyzed questionnaire data and the analyzed sleep disorder data yields a more accurate sleep disorder identification and diagnosis compared to either the analyzed questionnaire data or analyzed sleep disorder data alone.

In another embodiment of the invention, an apnea/hypopnea index is determined by an implantable medical device using implantably detected sensor data. A patient may also enter information related to, for example, age and health perceptions into an APM system or other user interface facility where the data may be analyzed or transmitted to the internal medical device for analysis. Based on the combination of the analyzed AHI data and patient-reported data, a severity or binary diagnosis of sleep apnea may be determined for the patient and data concerning same may be transmitted from the implantable medical device to a patient-external system for display to the patient and/or clinician.

According to various embodiments, analyzing device-acquired data and manually-reported patient data may involve weighting the data based on various factors, such as patient-specific risk factors and demographics. For example, if a patient has a high AHI but is not in an “at risk” demographic, based on analyzed manually-reported patient data, then when the analyzed results are combined, AHI data may be weighted less than if a patient were in an “at risk” demographic. This may result in a negative diagnosis for sleep apnea, or a degree of severity of apnea may be lower compared to an “at risk” patient.

Alternatively, if a patient has a low AHI but is in an “at risk” demographic, based on analyzed manually-reported patient data, the “at risk” demographic information may be given greater weight when the analyzed results are combined and a positive diagnosis for sleep apnea may be determined. Useful techniques for weighing various conditions and factors affecting a patient that may be implemented in the context of the present invention are disclosed in commonly owned U.S. patent application Ser. No. 10/643,016, filed Aug. 18, 2003 under Attorney Docket No. GUID.088PA, which is hereby incorporated herein by reference.

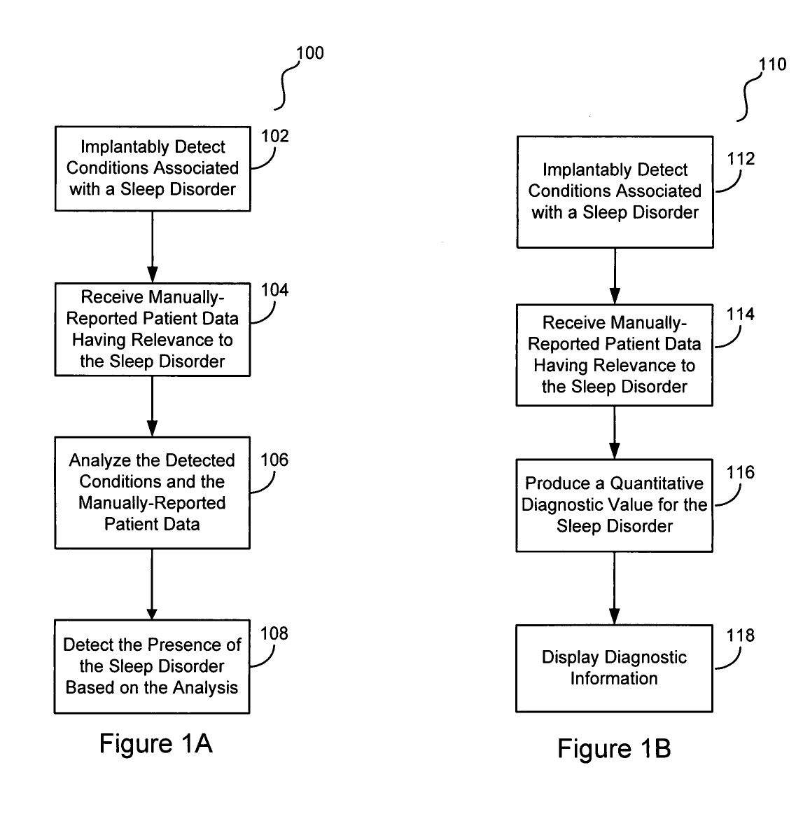

Referring now to FIG. 1A, a flowchart of a method for diagnosing sleep disorders is illustrated in accordance with embodiments of the present invention. According to method 100, conditions associated with a sleep disorder are implantably detected 102, and manually-reported patient data is received 104. The implantably detected conditions and the manually-reported patient data are analyzed 106, and the presence of a sleep disorder is detected 108 based on the analysis.

FIG. 1B is a flowchart of another method 110 for diagnosing sleep disorders according to other embodiments of the present invention. In method 110, data related to conditions associated with a sleep disorder is implantably detected 112, and manually-reported patient data is received 114. The implantably detected data and the manually-reported data are analyzed, and a quantitative diagnostic value for the sleep disorder is produced 116. Diagnostic information concerning the sleep disorder may be displayed 118 to the patient and/or clinician.

FIG. 1C is a flowchart of a method for diagnosing sleep disorders in accordance with further embodiments of the present invention. According to method 120, conditions associated with a sleep disorder are implantably sensed 122. A detection value is computed 124 based on the sensed conditions. Manually-reported patient data is received 126, and a patient data score is computed 128 using the manually-reported patient data. A quantitative diagnostic value for the sleep disorder is produced 130 using the detection value and the patient data score. The quantitative diagnostic value may be a numerical value, a Boolean value, or other value, character, or graphic indicative of the presence, absence, and/or severity of the sleep disorder.

The methods of FIGS. 1A-1C involve implantably detecting or sensing patient conditions associated with a sleep disorder. Such detection or sensing may be accomplished using a patient-internal medical device, such as an implantable cardiac rhythm management (CRM) or monitoring device. For example, characteristics associated with a patient's respiration, such as depth or shallowness of breathing, may be detected using a transthoracic impedance sensor integrated with or otherwise coupled to an implantable CRM or monitoring device.

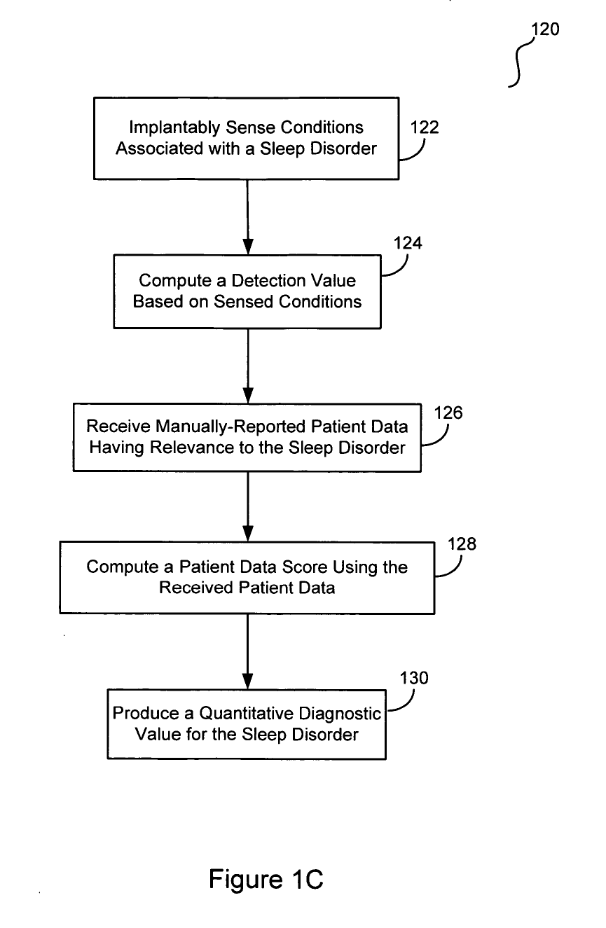

FIG. 2 illustrates a method for diagnosing sleep disorders according to embodiments of the present invention. Diagnostic data from a questionnaire 205 may be entered, and sleep disorder data 210 acquired by an implantable or partially implantable device may be generated. Each of the questionnaire and sleep disorder data may be analyzed, such as by comparing the data to respective thresholds as previously discussed. For example, questionnaire data 205 may be compared to the questionnaire threshold values 215, yielding a binary result, e.g., a presence or absence of a sleep disorder. Sleep disorder data 210 may be compared to sleep disorder threshold 220 value, also yielding a binary result.

The results of the threshold comparison operations may used jointly to make a combined decision 230 as the presence or absence of a sleep disorder. Alternatively, the diagnostic data 205 and sleep disorder data 210 may be analyzed, the data combined, and a composite index 235 result generated. The composite index 235 may show a patient's sleep disorder status on a graduated scale of severity, such as by use of a sleep disorder index, for example.

Although threshold testing of data associated with sleep disorders is described above, the present invention is not limited to arriving at a diagnosis using analyses that utilizes threshold comparison operations. Rather, other techniques or analyses may be used including Boolean, decision tree, neural networks, or other formulaic analysis methods to generate a binary diagnosis or a scale of sleep disorder severity.

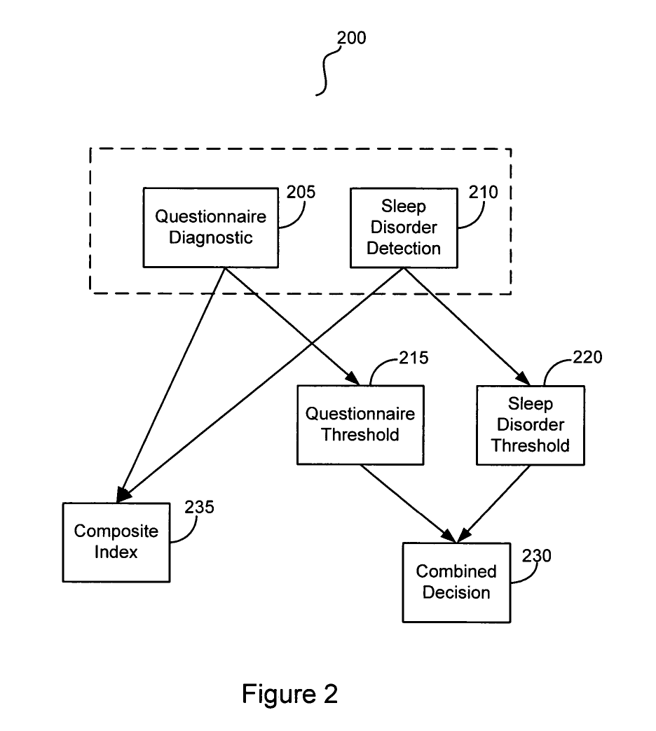

Embodiments of the invention may analyze multiple sets of data related to sleep disorder parameters. FIG. 3 illustrates a method 300 for combining multiple diagnostics to yield a single diagnostic output. According to FIG. 3, an apnea diagnostic 305, weight diagnostic 310, diet diagnostic 315, and diagnostic 4 320 may be combined using a method for combining results 325, such as the methods for combining data previously discussed. The combined results may yield a single diagnostic output 330, which may be a Boolean result or a numerical indicator, for example.

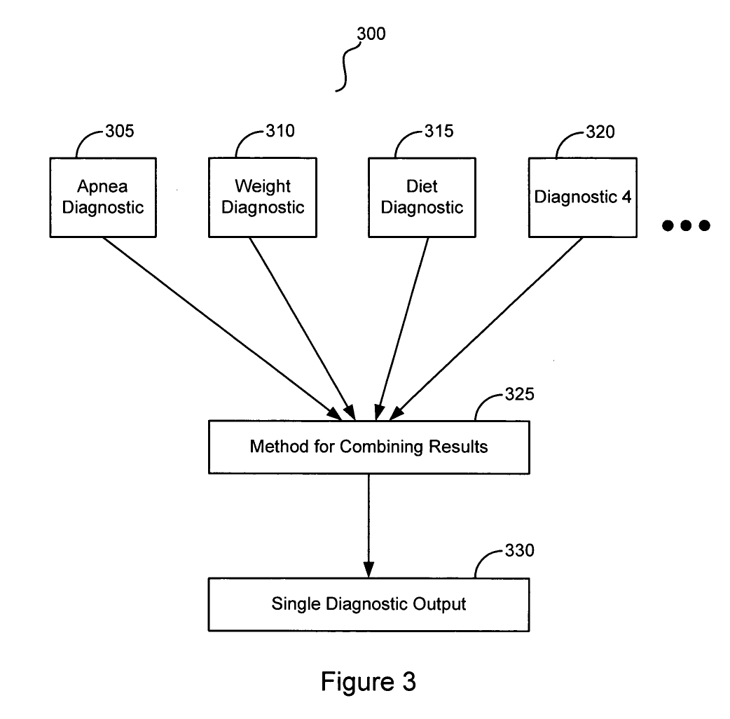

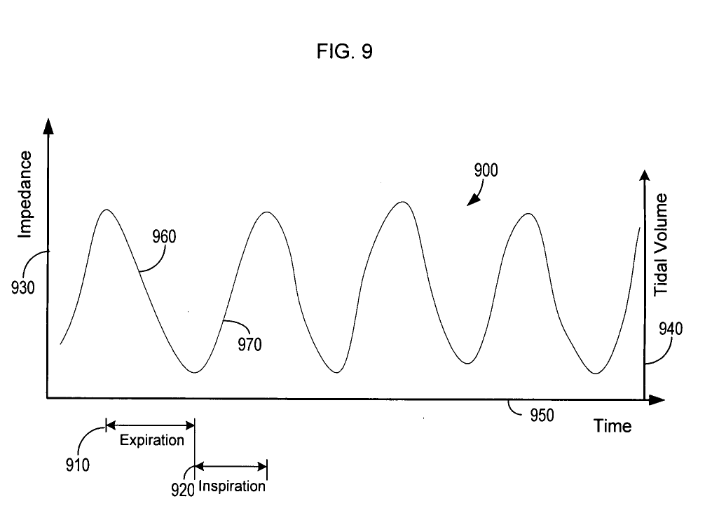

FIG. 4 is an illustration of a respiratory waveform 400 developed using a transthoracic impedance sensor of an implantable pacemaker. The waveform 400 represents one of several types of physiologic signal that may be used to sense or detect a sleep disorder. In this illustrative example, the respiratory waveform 400 is analyzed typically by pacemaker circuitry for purposes of detecting disordered breathing, such as apnea and hypopnea. Aberrations in the respiratory waveform 400 are analyzed to detect apnea and hypopnea on a per-hour basis so that an apnea-hypopnea index may be computed. FIGS. 9-12 and accompanying discussion describe detection and analysis of apnea and hypopnea in greater detail.

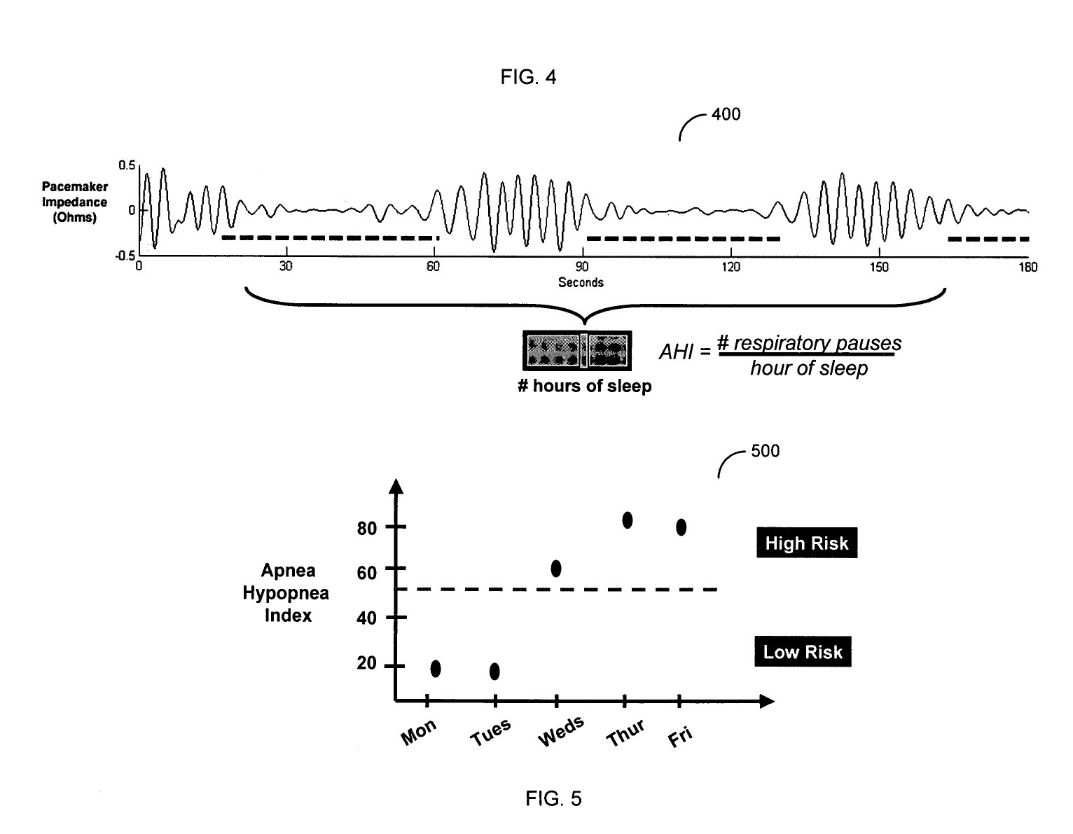

FIG. 5 illustrates AHI trend data 500 computed by use of an implantable medical device, such as a pacemaker, resynchronizer, or other cardiac monitoring or energy delivery device. One or both of the raw AHI data and the trend data developed from same may be computed by the implantable medical device. In one system configuration, for example, the AHI data may be acquired by the implantable medical device and transmitted to a patient-external system, and the AHI trend data 500 may be computed by the patient-external system for presentation to the clinician.

The AHI trend data 500 illustrated in FIG. 5 shows an AHI threshold at about 52 respiratory pauses per hour, below which the frequency of these events is considered low risk and above which the frequency of such events is considered high risk. The threshold may be adjusted by the patient's physician in a manner appropriate for the particular patient.

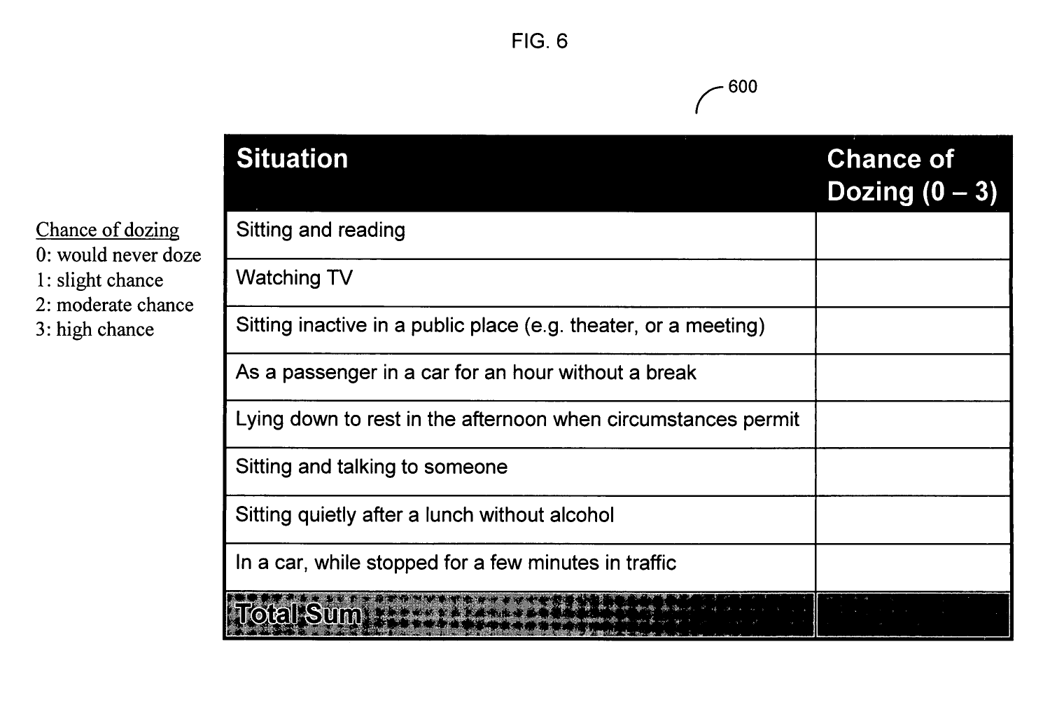

FIG. 6 is an example of a questionnaire by which patient condition information may be manually reported for entry as data into the diagnostics system. The questionnaire illustrated in FIG. 6 includes questions that are assigned numerical values. For example, an answer of 0 indicates that the patient would never doze during the particular situation identified in the questionnaire (e.g., Watching TV). An answer of 1 indicates a slight chance of dozing, an answer of 2 indicates a moderate chance of dozing, and an answer of 3 indicates a high chance of dozing during the particular situation identified in the questionnaire. The scores for the questions may be summed. The total score corresponds to a level of risk of a particular sleep disorder.

The questionnaire shown in FIG. 6 represents one of many different types of question/answer facilities that permits manually-reported patient data related to sleep disorders to be acquired. As previously discussed, the sleep disorder may be a disorder other than disordered breathing, including involuntary muscle movement disorders such as restless leg syndrome, periodic limb movement disorder, and bruxism, for example. Questions may be directed to a particular sleep disorder and, as such, the context of the questions may be focused accordingly.

The questionnaires may be developed to conform to established question/answer formats, such as an ESS format, or may be proprietary. Moreover, the questions may be developed in a manner that allows patient perception of conditions to be verified by the particular sensors or devices used to sense/detect the presence of particular sleep disorders. Contextual alignment between questions, patient answers, and physiologic sensor/device analysis capabilities may advantageously increase the speed and accuracy of sleep disorder diagnoses.

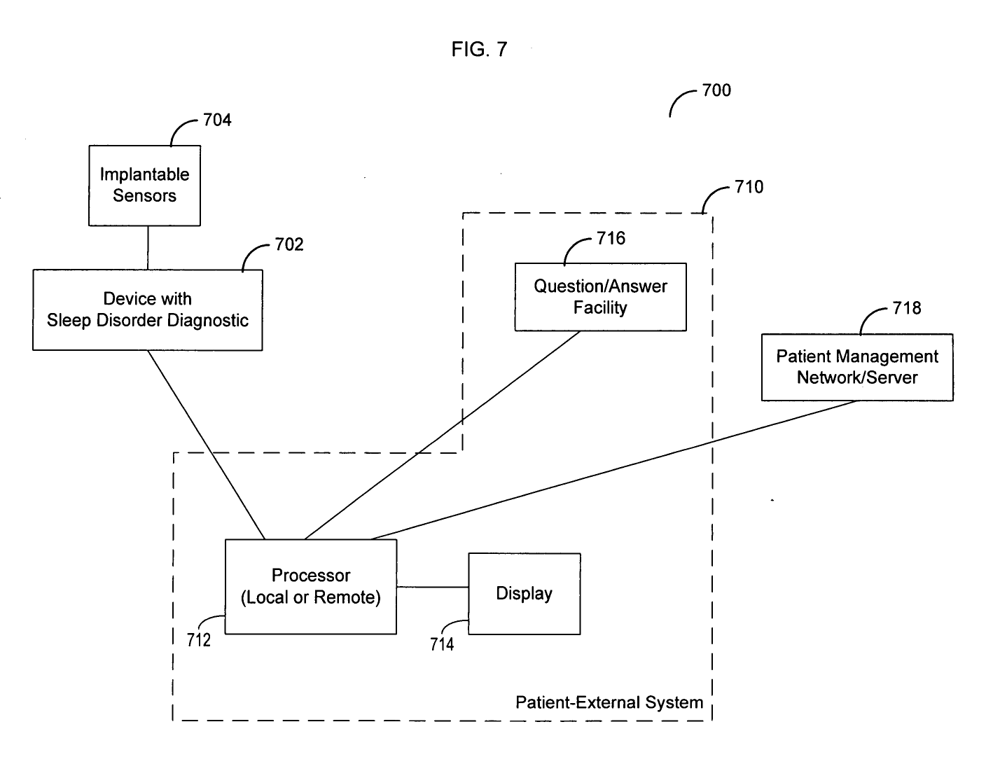

FIG. 7 is a block diagram of a diagnostic system according to an embodiment of the present invention. According to the embodiment shown in FIG. 7, a sleep disorder diagnostic system 700 includes one or more implantable sensors 704 that are configured to sense a physiologic parameter useful in detecting the presence of a particular sleep disorder. Although described generally as being implantable, it is understood that all or some of the sensor 704 may be patient-external sensors in certain embodiments. The sensors 704 are communicatively coupled to a device 702 that includes a sleep disorder diagnostic.

The device 702 may be implantable or patient-external. For example, the device 702 may be a cardiac rhythm management or monitoring system that incorporates a sleep disorder diagnostic. The device may also be a nerve stimulation device or a positive airway pressure device, for example. The device 702 may further be configured to deliver therapy to treat a sleep disorder. The sensors 704 may include one or more of transthoracic impedance sensors, EMG sensors, EEG sensors, cardiac electrogram sensors, nerve activity sensors, accelerometers, posture sensors, proximity sensors, electrooculogram (EOG) sensors, photoplethysmography sensors, blood pressure sensors, peripheral arterial tonography sensors, and/or other sensors useful in sensing conditions associated with sleep disorders.

The device 702 is configured to communicate with a patient-external system 710, which may be a programmer, home/bed-side system, or interface to a patient management network/sever 718, such as an advanced patient management system. The patient-external system 710 includes a processor 712 and is typically coupled to a display 714. A question/answer facility 716 is coupled to the processor 712.

The processor 712 is configured to receive manually-reported patient data from the question/answer facility 716 and sensor data from the device 702. The processor 712 operates on these data in a manner previously described to produce a diagnostic value or parameter indicative of the presence, absence, and/or severity of a sleep disorder. The sleep disorder diagnostic system 700 shown in FIG. 7 may be implemented in a variety of implantable or patient-external devices and systems, including cardiac monitoring or energy delivery devices, nerve stimulation devices, and positive airway pressure devices, among others.

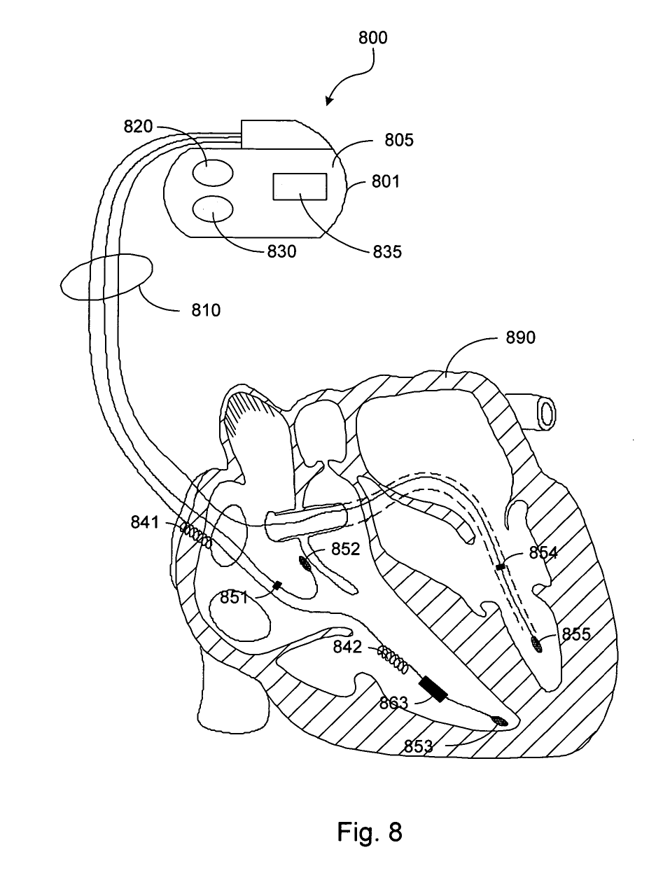

FIG. 8 is an illustration of a cardiac rhythm management system that implements sleep disorder diagnostics in accordance with an embodiment of the present invention. The system 800 shown in FIG. 8 may be configured to include circuitry and functionality for sleep disorder detection in accordance with embodiments of the invention. In this illustrative example, sleep disorder diagnostic circuitry 835 is configured as a component of a pulse generator 805 of a cardiac rhythm management device 800. The implantable pulse generator 805 is electrically and physically coupled to an intracardiac lead system 810. The sleep disorder diagnostic circuitry 835 may alternatively be implemented in a variety of implantable monitoring, diagnostic, and/or therapeutic devices, such as an implantable cardiac monitoring device, an implantable drug delivery device, or an implantable neurostimulation device, for example.

Portions of the intracardiac lead system 810 are inserted into the patient's heart 890. The intracardiac lead system 810 includes one or more electrodes configured to sense electrical cardiac activity of the heart, deliver electrical stimulation to the heart, sense the patient's transthoracic impedance, and/or sense other physiological parameters, e.g., cardiac chamber pressure or temperature. Portions of the housing 801 of the pulse generator 805 may optionally serve as a can electrode.

Communications circuitry is disposed within the housing 801, facilitating communication between the pulse generator 805 including the sleep disorder diagnostic circuitry 835 and an external device, such as a sleep disordered breathing therapy device, programmer, and/or APM system. The communications circuitry can also facilitate unidirectional or bidirectional communication with one or more implanted, external, cutaneous, or subcutaneous physiologic or non-physiologic sensors, patient-input devices and/or information systems.

The pulse generator 805 may optionally incorporate a EMG sensor 820 disposed on the housing 801 of the pulse generator 805. The EMG sensor may be configured, for example, to sense myopotentials of the patient's skeletal muscle in the pectoral region. Myopotential sensing may be used in connection with sleep disorders associated with involuntary limb movement as previously discussed.

The pulse generator 805 may further include a sensor configured to detect patient motion. The motion detector may be implemented as an accelerometer positioned in or on the housing 801 of the pulse generator 805. If the motion detector is implemented as an accelerometer, the motion detector may also provide acoustic information, e.g. rales, coughing, S1-S4 heart sounds, cardiac murmurs, and other acoustic information.

The lead system 810 of the CRM device 800 may incorporate a transthoracic impedance sensor that may be used to acquire the patient's cardiac output, or other physiological conditions related to the patient's sleep disorder(s). The transthoracic impedance sensor may include, for example, one or more intracardiac electrodes 840, 842, 851-855, 863 positioned in one or more chambers of the heart 890. The intracardiac electrodes 841, 842, 851-855, 861, 863 may be coupled to impedance drive/sense circuitry 830 positioned within the housing of the pulse generator 805.

The impedance signal may also be used to detect the patient's respiration waveform and/or other physiological changes that produce a change in impedance, including pulmonary edema, heart size, cardiac pump function, etc. The respiratory and/or pacemaker therapy may be altered on the basis of the patient's heart condition as sensed by impedance.

In one example, the transthoracic impedance may be used to detect the patient's respiratory waveform, examples of which are shown in FIGS. 9-12. A voltage signal developed at the impedance sense electrode 852, illustrated in FIG. 9, is proportional to the patient's transthoracic impedance and represents the patient's respiration waveform. The transthoracic impedance increases during respiratory inspiration and decreases during respiratory expiration. The transthoracic impedance may be used to determine the amount of air moved in one breath, denoted the tidal volume and/or the amount of air moved per minute, denoted the minute ventilation. A normal “at rest” respiration pattern, e.g., during non-REM sleep, includes regular, rhythmic inspiration—expiration cycles without substantial interruptions, as indicated in FIG. 9.

Returning to FIG. 8, the lead system 810 may include one or more cardiac pace/sense electrodes 851-855 positioned in, on, or about one or more heart chambers for sensing electrical signals from the patient's heart 890 and/or delivering pacing pulses to the heart 890. The intracardiac sense/pace electrodes 851-855, such as those illustrated in FIG. 8, may be used to sense and/or pace one or more chambers of the heart, including the left ventricle, the right ventricle, the left atrium and/or the right atrium. The lead system 810 may include one or more defibrillation electrodes 841, 842 for delivering defibrillation/cardioversion shocks to the heart.

The pulse generator 805 may include circuitry for detecting cardiac arrhythmias and/or for controlling pacing or defibrillation therapy in the form of electrical stimulation pulses or shocks delivered to the heart through the lead system 810. Sleep disorder diagnostic circuitry 835 may be housed within the housing 801 of the pulse generator 805. The sleep disorder diagnostic circuitry 835 may be coupled to various sensors, including the transthoracic impedance sensor 830, EMG sensor 820, EEG sensors, cardiac electrogram sensors, nerve activity sensors, and/or other sensors capable of sensing physiological signals useful for sleep disorder detection.

The sleep disorder diagnostic circuitry 835 may be coupled to a sleep disorder detector configured to detect sleep disorders such as disordered breathing, and/or movement disorders. An arousal detector and a sleep disorder detector may be coupled to a processor that may use information from the arousal detector and the sleep disorder detector to associate sleep disorder events with arousal events. The processor may trend the sleep disorder events and/or arousal events, associate the sleep disorder events with arousal events, and/or use the detection of the arousal events and/or the sleep disorder events for a variety of diagnostic purposes. The sleep disorder detector and/or the processor may also be configured as a component of the pulse generator 805 and may be positioned within the pulse generator housing 801. In one embodiment, information about the sleep disorder events and/or arousal events may be used to adjust therapy delivered by the CRM device 800 and/or other therapy device.

Referring now to FIGS. 9-12, several respiration waveforms are shown that may be developed by a medical device implementing a sleep disorder detection methodology of the present invention. With reference to FIG. 9, an impedance signal 900 is illustrated, which is useful for determining sleep, sleep state, and sleep disordered breathing. The impedance signal 900 may be developed, for example, from an impedance sense electrode in combination with a CRM device. The impedance signal 900 is proportional to the transthoracic impedance, illustrated as an Impedance 930 on the abscissa of the left side of the graph in FIG. 9.

The impedance 930 increases 970 during any respiratory inspiration 920 and decreases 960 during any respiratory expiration 910. The impedance signal 900 is also proportional to the amount of air inhaled, denoted by a tidal volume 940, illustrated on the abscissa of the right side of the graph in FIG. 9. The variations in impedance during respiration, identifiable as the peak-to-peak variation of the impedance signal 900, may be used to determine the respiration tidal volume 940. Tidal volume 940 corresponds to the volume of air moved in a breath, one cycle of expiration 910 and inspiration 920. A minute ventilation may also be determined, corresponding to the amount of air moved per a minute of time 950 illustrated on the ordinate of the graph in FIG. 9.

Breathing disorders may be determined using the impedance signal 930. During non-REM sleep, a normal respiration pattern includes regular, rhythmic inspiration—expiration cycles without substantial interruptions. When the tidal volume of the patient's respiration, as indicated by the transthoracic impedance signal, falls below a hypopnea threshold, then a hypopnea event is declared. For example, a hypopnea event may be declared if the patient's tidal volume falls below about 50% of a recent average tidal volume or other baseline tidal volume value. If the patient's tidal volume falls further to an apnea threshold, e.g., about 10% of the recent average tidal volume or other baseline value, an apnea event is declared.

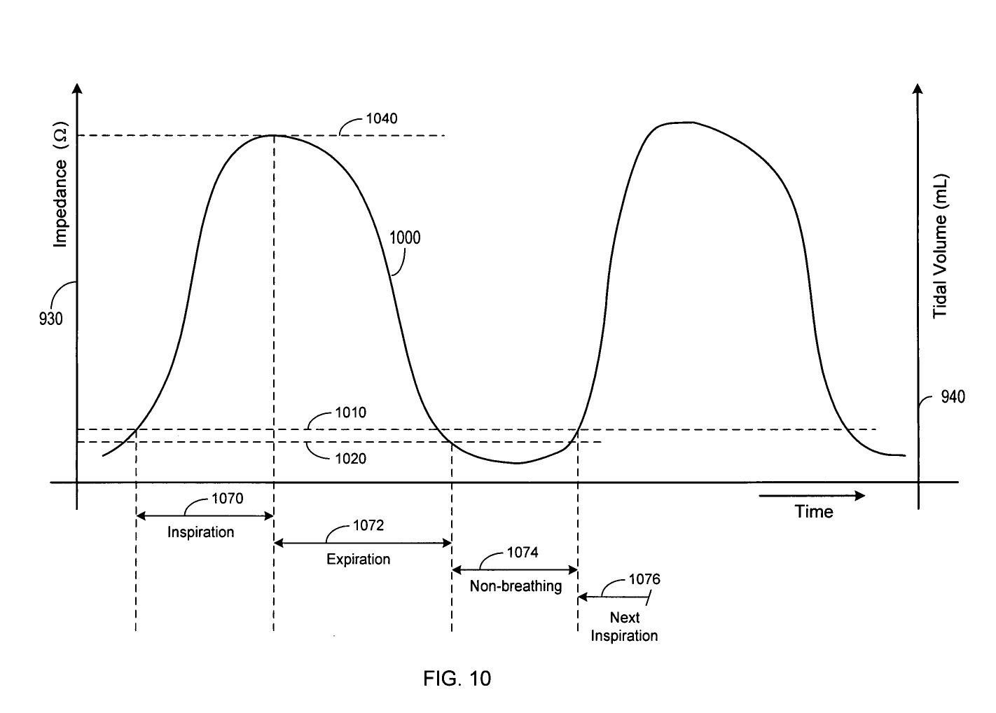

FIGS. 10-12 are graphs of transthoracic impedance and tidal volume, similar to FIG. 9 previously described. As in FIG. 9, FIGS. 10-12 illustrate the impedance signal 1000, 1100, 1200 proportional to the transthoracic impedance, again illustrated as impedance 930 on the abscissa of the left side of the graphs in FIGS. 10-12. The impedance 930 increases during any respiratory inspiration and decreases during any respiratory expiration. As before, the impedance signal 1000, 1100, 1200 is also proportional to the amount of air inhaled, denoted the tidal volume 940, illustrated on the abscissa of the right side of the graph in FIGS. 10-12. The magnitude of variations in impedance and tidal volume during respiration are identifiable as the peak-to-peak variation of the impedance signal 1000, 1100, 1200.

FIG. 10 illustrates respiration intervals used for disordered breathing detection useful in accordance with embodiments of the invention. Detection of disordered breathing may involve defining and examining a number of respiratory cycle intervals. A respiration cycle is divided into an inspiration period corresponding to the patient inhaling, an expiration period, corresponding to the patient exhaling, and a non-breathing period occurring between inhaling and exhaling.

Respiration intervals are established using an inspiration threshold 1010 and an expiration threshold 1020. The inspiration threshold 1010 marks the beginning of an inspiration period 1070 and is determined by the transthoracic impedance signal 1000 rising above the inspiration threshold 1010. The inspiration period 1070 ends when the transthoracic impedance signal 1000 is a maximum 1040. The maximum transthoracic impedance signal 1040 corresponds to both the end of the inspiration interval 1070 and the beginning of an expiration interval 1072. The expiration interval 1072 continues until the transthoracic impedance 1000 falls below an expiration threshold 1020. A non-breathing interval 1074 starts from the end of the expiration period 1072 and continues until the beginning of a next inspiration period 1076.

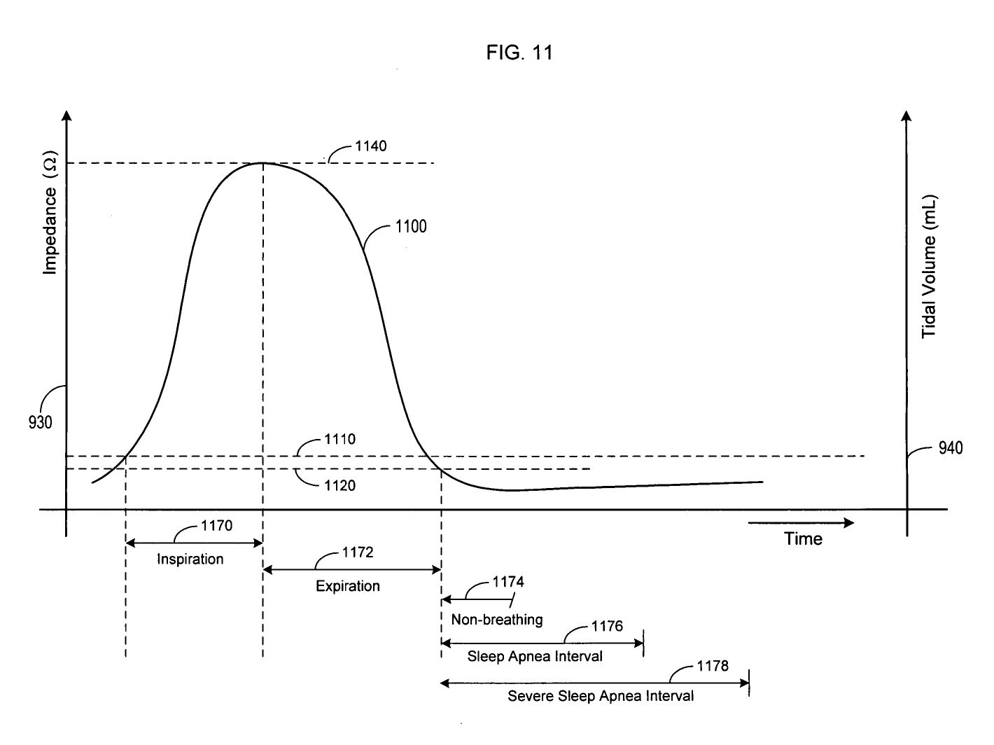

Detection of sleep disordered breathing events such as sleep apnea and severe sleep apnea is illustrated in FIG. 11. The patient's respiration signals are monitored and the respiration cycles are defined according to an inspiration 1170, an expiration 1172, and a non-breathing 1174 interval as described in connection with FIG. 10. A condition of sleep apnea is detected when a non-breathing period 1174 exceeds a first predetermined interval 1176, denoted the sleep apnea interval. A condition of severe sleep apnea is detected when the non-breathing period 1174 exceeds a second predetermined interval 1178, denoted the severe sleep apnea interval. For example, sleep apnea may be detected when the non-breathing interval exceeds about 10 seconds, and severe sleep apnea may be detected when the non-breathing interval exceeds about 20 seconds.

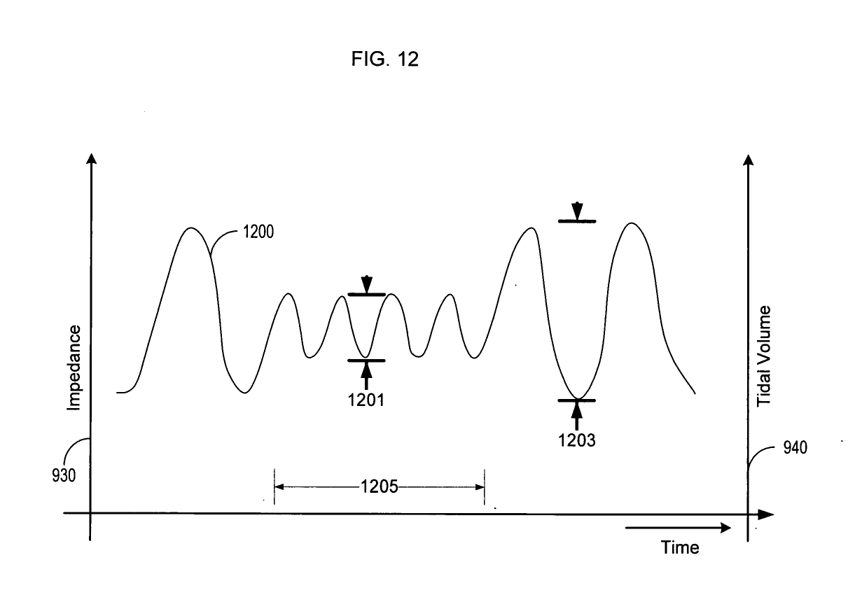

Hypopnea is a condition of sleep disordered breathing characterized by abnormally shallow breathing. FIG. 12 is a graph of tidal volume derived from transthoracic impedance measurements. The graph of FIG. 12 illustrating the tidal volume of a hypopnea episode may be compared to the tidal volume of a normal breathing cycle illustrated previously in FIG. 9, which illustrated normal respiration tidal volume and rate. As shown in FIG. 12, hypopnea involves a period of abnormally shallow respiration, possible at an increased respiration rate.

Hypopnea is detected by comparing a patient's respiratory tidal volume 1203 to a hypopnea tidal volume 1201. The tidal volume for each respiration cycle may be derived from transthoracic impedance measurements acquired in the manner described previously. The hypopnea tidal volume threshold may be established by, for example, using clinical results providing a representative tidal volume and duration of hypopnea events. In one configuration, hypopnea is detected when an average of the patient's respiratory tidal volume taken over a selected time interval falls below the hypopnea tidal volume threshold. Furthermore, various combinations of hypopnea cycles, breath intervals, and non-breathing intervals may be used to detect hypopnea, where the non-breathing intervals are determined as described above.

In FIG. 12, a hypopnea episode 1205 is identified when the average tidal volume is significantly below the normal tidal volume. In the example illustrated in FIG. 12, the normal tidal volume during the breathing process is identified as the peak-to peak value identified as the respiratory tidal volume 1203. The hypopnea tidal volume during the hypopnea episode 1205 is identified as hypopnea tidal volume 1201. For example, the hypopnea tidal volume 1201 may be about 50% of the respiratory tidal volume 1203. The value 50% is used by way of example only, and determination of thresholds for hypopnea events may be determined as any value appropriate for a given patient. In the example above, if the tidal volume falls below 50% of the respiratory tidal volume 1203, the breathing episode may be identified as a hypopnea event, originating the measurement of the hypopnea episode 1205.

Sleep disorder detection according the present invention may employ a wide variety of sensors, implantable and non-implantable medical devices, systems and interfaces for acquiring manually-reported patient data, sleep disorder detection techniques, and therapies to treat sleep disorders. The embodiments discussed herein represent several non-limiting illustrative implementations.

These and other implementations for detecting conditions associated with sleep disorders may also provide for detection that a patient is asleep. A method of sleep detection is described in commonly owned U.S. patent application Ser. No. 10/309,771, filed Dec. 4, 2002, which is incorporated herein by reference in its entirety. In addition, classification of sleep state, including classification of rapid eye movement sleep (REM sleep) and non-REM sleep may also be used to enhance sleep detection and/or to determine the duration of various sleep states. Sensing abnormal sleep state durations may be indicative of restless sleep due to sleep apnea for example. Methods and systems involving classifying the patient's sleep state are described in commonly owned U.S. patent application Ser. No. 10/643,006, filed Aug. 18, 2003 under Attorney Docket No. GUID.060PA, which is hereby incorporated herein by reference.

Detection of a sleep disorder may involve detecting one or more conditions indicative of sleep disordered breathing (SDB). Methods and systems for detection and treatment of disordered breathing is described in commonly owned U.S. patent application Ser. No. 10/643,203, filed Aug. 18, 2003 under Attorney Docket No. GUID.059PA, which is hereby incorporated herein by reference. Another implementation of SDB detection includes detection and analysis of respiratory waveform patterns. Methods and systems for detecting disordered breathing based on respiration patterns are more fully described in commonly owned U.S. patent application Ser. No. 10/309,770, filed Dec. 4, 2002 under Attorney Docket No. GUID.054PA and U.S. patent application Ser. No. 10/309,771, filed Dec. 4, 2002 under Attorney Docket No. GUID.064PA, which are hereby incorporated herein by reference.

Detection of a sleep disorder may also involve detecting one or more conditions relating to involuntary muscle movement disorders, such as restless leg syndrome, periodic limb movement disorder, and bruxism, for example. Systems and techniques for detecting involuntary muscle movement disorders that may be implemented in accordance with the present invention are disclose in commonly owned U.S. patent application Ser. No. 10/920,675, filed Aug. 17, 2004 under GUID.106PA; Ser. No. 10/939,834, filed Sep. 13, 2004 under Attorney Docket No. GUID.127PA; and Ser. No. 10/939,639, filed Sep. 13, 2004 under Attorney Docket No. GIUD.141PA, all of which are hereby incorporated herein by reference.

Various modifications and additions may be made to the embodiments discussed herein without departing from the scope of the present invention. In some configurations, for example, implantable or partially implantable sensors that sense sleep disorder conditions may be used in combination with a patient-implantable medical device or a patient-external medical device. In other configurations, patient-external sensors that sense sleep disorder conditions may be used in combination with a patient-implantable medical device or a patient-external medical device. A wide variety of sensor and medical device configurations that provide for the integration of sleep disorder sensor data and manually-reported patient data to produce diagnostic information concerning the sleep disorder are contemplated. Accordingly, the scope of the present invention should not be limited by the particular embodiments described above, but should be defined only by the claims set forth below and equivalents thereof.

1. A method, comprising:
implantably sensing one or more conditions associated with a sleep disorder;
computing a detection value based on the sensed one or more conditions;
receiving manually-reported patient data having relevance to the sleep disorder or patient condition;
computing a patient data score using the received patient data; and
producing a diagnostic value for the sleep disorder using the detection value and patient data score.

2. The method of claim 1, wherein the diagnostic value is indicative of presence or non-presence of the sleep disorder.

3. The method of claim 1, wherein the diagnostic value is indicative of a level of severity of the sleep disorder.

4. The method of claim 1, wherein the diagnostic value is a Boolean value.

5. The method of claim 1, wherein the diagnostic value is a numerical value.

6. The method of claim 1, wherein producing the diagnostic value comprises logically combining the detection value and the patient data score to produce the diagnostic value.

7. The method of claim 1, wherein producing the diagnostic value comprises mathematically combining the detection value and the patient data score to produce the diagnostic value.

8. The method of claim 1, wherein receiving the patient data comprises receiving the patient data in the form of a questionnaire, the questionnaire comprising a plurality of questions each of which is assigned a value, and computing the patient data score comprises operating on the values to compute the patient data score.

9. The method of claim 1, wherein computing the detection value comprises summing a number of the one or more conditions sensed over a predefined duration of time.

10. The method of claim 1, wherein:
sensing the one or more conditions further comprises detecting apnea or hypopnea events over a predefined duration of time; and
computing the detection values comprises computing an apnea/hypopnea index (AHI) based on a number of the apnea or hypopnea events detected over the predefined duration of time.

11. The method of claim 1, further comprising:
receiving one or more patient condition indicators; and
producing one or more patient condition values corresponding to the one or more patient condition indicators;
wherein the diagnostic value is produced using the detection value, patient data score, and the one or more patient condition values.

12. The method of claim 1, further comprising providing a first threshold associated with detection of the one or more conditions associated with the sleep disorder, and providing a second threshold associated with the patient data, wherein:
the detection value is computed using the sensed one or more conditions that exceed the first threshold; and
the patient data score is computed using the received patient data that exceeds the second threshold.

13. The method of claim 1, further displaying one or more of the diagnostic value, patient data score, and detection data.

14. The method of claim 1, further comprising:
producing trend data using a plurality of the detection values computed over time; and
producing alert information when the trend data exceeds one or more thresholds indicative of presence of the sleep disorder.

15. A method for evaluating sleep disorders, comprising:
implantably detecting one or more conditions associated with a sleep disorder;
receiving manually-reported patient data having relevance to the sleep disorder or patient condition; and
producing a quantitative diagnostic value for the sleep disorder using the detected conditions and the patient data.

16. The method of claim 15, wherein producing the diagnostic value is performed by a networked system.

17. The method of claim 15, wherein receiving the patient data comprises receiving the patient data via a programmer or a patient management system interface.

18. The method of claim 15, wherein the diagnostic value comprises a Boolean value developed from a Boolean operation performed on the patient data and data indicative of the sensed one or more conditions associated with the sleep disorder.

19. An apparatus, comprising:
a body implantable sensing device configured to sense one or more conditions associated with a sleep disorder;
a user interface device configured to receive manually-reported patient data having relevance to the sleep disorder or patient condition; and
a processing system configured to compute a detection value based on the sensed one or more conditions, compute a patient data score using the received patient data, and produce a diagnostic value for the sleep disorder using the detection value and patient data score.

20. The apparatus of claim 19, wherein the body implantable sensing device comprises an implantable cardiac monitoring device or an implantable cardiac energy delivery device.

21. The apparatus of claim 19, wherein the body implantable sensing device comprises a sleep disordered breathing sensor, and the detection value computed by the processing system comprises an apnea/hypopnea index.

22. The apparatus of claim 19, wherein the user interface device comprises a programmer configured to communicatively couple to the body implantable sensing device.

23. The apparatus of claim 19, wherein the user interface device comprises a network interface configured to communicatively couple to a patent management network system.
